(19) United States
(12) Patent Application Publication
(10) Pub. No.: US 2003/0139666 A1
(43) Pub. Date: Jul. 24, 2003

(54) Method and device for the stroboscopic recording and reproduction of repetitive processes

(76) Inventors: Holger Klemm, Bradenburg (DE); Klaus Ginter, Hohen Neuendorf (DE); Reinhard Tietze, Hohen Neuendorf (DE)

Correspondence Address: DENNISON, SCHULTZ & DOUGHERTY, 1745 JEFFERSON DAVIS HIGHWAY, ARLINGTON, VA, 22202, US

(21) Appl. No.: 10/204,411

(22) PCT Filed: Mar. 5, 2001
(86) PCT No.: PCT/EP01/02477

(30) Foreign Application Priority Data
Mar. 3, 2000 (DE) 10009981.5
Apr. 7, 2000 (DE) 10017162.1

(51) Int. Cl. A61B 5/05

(52) U.S. Cl. 600/410

(57) ABSTRACT

The invention relates to a method and to a device for the stroboscopic recording and reproduction of images of a repetitive process (12), especially of moving vocal chords (larynx diagnostics). The aim of the invention is to improve image quality, especially the brightness and definition of the images while requiring little lighting equipment. According to the inventive method, the process (12) to be observed is illuminated with a steady light source (14). Trigger pulses S_{Trigger} are generated to trigger exposures to record the image information of the process (12) by an image sensor (16). The image information from a plurality of subsequent exposures is added in a back-ground memory of the image sensor (16) to produce image information sums. The image information sums thus obtained are stored in the background memory (18) of the image sensor (16) and the background memory (18) is read and deleted, and the image information sums are processed to give a video signal.

The invention relates to a method and a device for the stroboscopic recording and reproduction of a repetitive process, especially moving vocal chords (larynx diagnostics). The invention is also references a device for the stroboscopic recording and reproduction of images of a repetitive process, especially moving vocal chords (larynx diagnostics).

Stroboscopic recordings of periodically or also non-periodically repetitive processes can be used for example to analyze the processes themselves or to observe slower events that are superimposed to the processes. For this purpose, synchronous to the processes that are to be observed recordings are made with the effect that the moving object is always recorded in the same phase position of the motion and thus appears to be stationary. Almost periodic processes can additionally be observed at drastically reduced motions when the recordings are performed with a frequency that is slightly shifted from the momentary frequency of the process.

For the examination of periodically or also non-periodically repetitive processes with the help of the stroboscopic effect currently two different technologies are known and implemented.

On one hand, there is the generation of high-speed flashes, which illuminate the object that is to be observed synchronously to its motion for a very brief time, and on the other hand there is the interruption of the light beam path of a steady light source with the help of a so-called shutter, which interrupts the light path between the steady light source and the observed object or between the observed object and the observer synchronously to the motion of the object that is to be observed.

Since the present invention is aimed particularly, but not exclusively at the use of larynx diagnostics, the following explanations are also based to a large extent on examples in this field. Larynx diagnostics here represents the examination of the vocal chords of a patient. This includes in particular the analysis of the moving vocal chords with the help of the stroboscopic effect.

In today's flash stroboscopes, the flashes can be triggered as a function of the patient's voice. For this purpose a microphone is arranged in the vicinity of the patient, usually close to the larynx. A conventional camera, generally a CCD camera, then records the moving vocal chords, independent from the voice.

With the help of a circuit, flashes are triggered such that the moving vocal chords are always only illuminated when the vocal chords have reached a certain aperture state (a certain phase). For the depiction of non-moving vocal chords, the flash always occurs at the same aperture state. For a drastically retarded motion, the phase of the light flashes is shifted minimally with each voice base frequency period.

Since the light flashes of existing flash stroboscopes are triggered only by certain phase conditions of the fundamental voice wave, illumination for not approximately periodically intonated sounds or without voice intonation flickers very much or is not possible. For this reason, conventional flash stroboscopes include apart from the flashlamp an additional steady light source for the vibration analysis, which serves the voice-independent illumination. The use of these two lamps results in the following problem for the diagnosis of vocal chords:

The flashlamp is of the XENON type for interrupted cold light of high power, while the steady light lamp supplies uninterrupted light of lower power.

The steady light lamp is generally a HALOGEN type lamp, sometimes a XENON type lamp (different from the flash XENON type). Due to these differing technologies, both lamps illuminate the vocal chords with light of different color temperatures. The different images of the vocal chords generated by this are a source for problems in the diagnosis.

For the purpose of partial compensation of these color temperature problems, in practice already electronic compensating circuits are used, which further increase expenses. Additionally color filters are used in front of the lamps, which are meant to adjust the color temperatures. The change-over processes between flash and steady light when using or discontinuing the use of the voice however basically lead to a considerable and disruptive flickering of the video image. Since the brightness control that is common for CCD cameras cannot influence the flash and thus can vibrate, it is also switched off when turning on the flash, which can cause additional over-exposure of the image. The examination especially of patients who can intonate only very briefly is impaired by these image interferences and also represents a difficulty for the examining physician.

Compared to steady light lamps, flashlamps additionally have only a fraction of the service life and cause more than ten times the cost of existing halogen steady light lamps.

Another disadvantage is the length of the individual flashes, which cannot be predicted with accuracy. Even in the case of periodic processes, this creates at least slightly flickering images.

So as to avoid the disadvantages of flash stroboscopy, color distortion, increased expenses and flickering we know for example from DE 43 09 353 C2 or EP 0 865 759 A1 of devices, which generate the stroboscopic effect with permanent illumination, but by selecting an electronic flap (shutter) of a video camera, as a function of the base frequency of the approximately periodically moving object that is to be observed. For performing larynx diagnostics, a microphone is also placed in the vicinity of the patient, generally close to the larynx. In order to observe periodically or also non-periodically repetitive processes other sensors, which supply electric starting signals that are synchronous to the fundamental wave, are also feasible.

Existing video cameras supply a sequential video signal that is divided into images and/or frames with a fixed image or frame frequency during the continuous operation of the video camera. In the following no differentiation is made between images and frames since this differentiation is not significant for the present invention. In the place of these two terms, the term image period is used in the following, which signifies the time between the beginning of a frame synchronizing signal and the subsequent frame synchronizing signal of the video standard that is employed. The term image signifies image information in the following, which exists when reading the image sensor at the output of the image sensor.

Image sensors in video cameras exhibit a multitude of image segments, which integrate the incident light intensity during an exposure time. At the end of the exposure time, the image segments forward the information of the integrated light intensities parallel to an intermediate memory, which is described in the following as back-ground memory. Until the next light intensities are passed by the image segments to the intermediate storage, the memory is being read and the video signal is generated. The exposure time of the image segments is synchronized with the camera's image frequency in conventional cameras. The end of the exposure time, which maximally lasts one image period, thus coincides with the end of the respective image period Tᵥ of both the image segments and of the back-ground memory.

In order to generate the stroboscopic effect under steady light and with a video camera it is possible to open and close the electronic flap of conventional image sensors in place of the flash synchronously with the process that is to be observed.

The devices from DE 43 09 353 C2 as well as EP 0 865 759 A1 each perform one exposure per reading process of the image sensor. The device from DE 43 09 353 C2 performs the reading process of the image sensor synchronously with the image frequency of the camera, while the device from EP 0 865 759 A1 performs the reading process of the image sensor directly after the exposure, i.e. not synchronously with the image period of the camera, but synchronously with the fundamental wave of the repetitive process. The latter therefore requires the additional expenses of separate writing and reading memory devices, which convert the video signal, which is not synchronous with the camera, into the video standard that is used.

In both devices from DE 43 09 353 C2 as well as EP 0 865 759 C1 the limitation to exactly one exposure leads to considerable demands that are placed on the light power of the exposure.

Another attempt to overcome the disadvantages of the existing flash stroboscopy are represented by the larynx stroboscopes offered by ALPHATRON, Rotterdam, Netherlands (e.g. www.dpmedicalsys.com), which operate with steady light sources.

The ALPHATRON devices however exhibit the following disadvantages:

the necessity for a very powerful steady light source (existing ALPHATRON stroboscopes have 300 Watt XENON light sources),

the higher price compared to HALOGEN lamps or the weaker XENON lamps, the higher power consumption as well as higher mass and larger volume of the powerful XENON light sources, flickering when using and discontinuing the use of the voice, presumably due to lacking trigger signals during the change-over processes from stroboscope operation without trigger signals to stroboscope operation with trigger signals.

A device and a method especially for larynx stroboscopy with continuous illumination can also be viewed in the brochure “Ent Endoscopy” by the applicant, which was offered at the MEDICA '99 trade show in November 1999. The brochure reveals that the stroboscopic effect is shifted into a CCD sensor. Detailed information about the method and design of the device is not revealed in the brochure.

The invention is based on the task of further developing a method and a device of the previously described kind in such a way that the image quality, particularly its brightness and definition are improved while requiring little lighting equipment. Additionally, the image interference occurring with all existing devices, such as flickering and over-exposure, when switching between the stroboscope and normal camera operation is supposed to be eliminated.

Pursuant to the invention, the problem is resolved on one hand with a method that includes the features of patent claim 1 and on the other hand with a device with the features described in claim 10.

Pursuant to the invention, the image sensor itself is used for intermediate storage of the image information as well as integrating (adding) the image information from several successive exposures in the image sensor itself. Compared to the state of the art, this new technology enables several exposures synchronous to the phenomenon before reading an image from the sensor.

Based on a particularly preferred embodiment, the recorded image information during the first image period is added n-fold with n>1 in the back-ground memory and stored, wherein the image information sum stored in the back-ground memory is read as reaction to a subsequent, second image period. For example for the video standard PAL with an image period of 20 ms and the extreme case of a base frequency of the process F=1 kHz and/or the base period 1 ms this results in twenty synchronous exposures before one reading occurs. For the same length of one individual exposure, i.e. also same pulse-duty factor and same image definition, as is the case in FR 2 761,171 A1 (=EP 0 865 759 A1) or DE 34 09 353 C2, this means twenty times the brightness. Relatively low frequencies such as F=100 Hz (T=10 ms) result e.g. in the case of PAL still in twice the brightness since two exposures can take place instead of only one exposure.

In another preferred embodiment, the recorded image information during several image periods is added n-fold with n>1 in the back-ground memory and stored, wherein the image information sum stored in the back-ground memory is read as reaction to the end of the n-th memory process. This basically results in a frequency-independent n-fold brightness with equal image definition compared to EP 0 865 759 A1 or DE 43 09 353 C2.

Furthermore it is provided that the brightness of the images of the video signal or of the image information sums read from the back-ground memory is measured, wherein the measured brightness is compared to a specified, desired brightness and wherein as a function of the comparison a total exposure time of all exposures, which are to be performed between two trigger processes of the image sensor, is established. Pursuant to another method of the invention, the exposure times of the individual image information that is to be added vary as a function of the established total exposure time and a momentary frequency that is deduced from the process. It has proven particularly useful to measure the actual total exposure time and compare it to the specified total exposure time, wherein the start of an exposure as well as the subsequent adding and storage processes are blocked when the specified total exposure time has been reached, and the blockage of the exposure and the subsequent adding and storage processes was eliminated as soon as the next reading process of the back-ground memory has been completed. The actual total exposure time at a certain point in time, here as well as in the following, represents the sum of all exposure times of the exposures that were performed between the previous reading process of the image sensor until now. The additional circuits for image brightness measurement and calculation of the exposure times of the individual exposures that are to be added enable consistent brightness as well as definition, independent from the voice frequency.

Another particularly preferred method is characterized by the fact that the trigger pulses are generated synchronously or asynchronously to the process or independently from the process so that the specified total exposure time can be reached. Due to the method suggested here, which guarantees the desired total exposure time even in the case of lacking or aperiodic vocal chord vibrations, the drastic flickering when using or discontinuing the use of the voice or with aperiodic voices is eliminated compared to the ALPHATRON device.

The considerably higher brightness despite equal illumination means that when employing this technology the required illumination power is at most one twentieth with equal image definition to EP 0 865 759 A1 or DE 43 09 353 C2. This reduction in the required light power enables the use of 150 Watt HALOGEN lamps as the steady light source for the stroboscopic diagnosis of the vibrating vocal chords even at higher frequencies up to 1 kHz.

Compared to the existing state of the art, the method described in the invention as well as the device provide always flicker-free images of consistent brightness, which facilitates the examining physician's work considerably.

Further details, benefits and features of the invention result not [only] from the claims, the features revealed in them—either alone and/or in combination—but also from the following description of one particularly preferred example, which is shown in the drawing.

It shows:

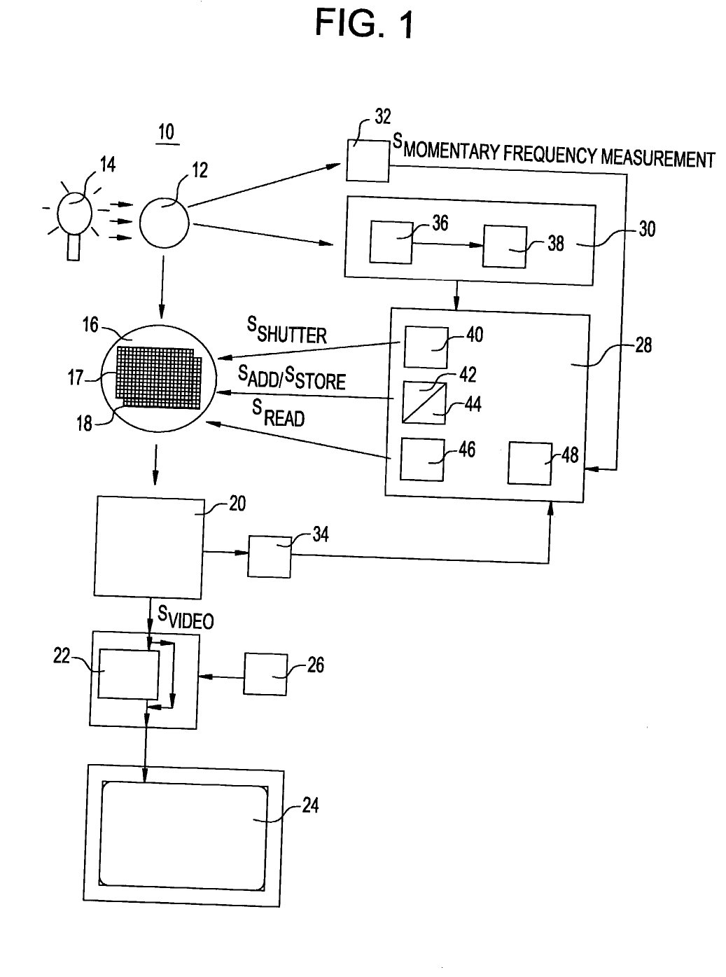

FIG. 1 a diagrammatic set-up of a device for the stroboscopic recording and reproduction of repetitive processes,

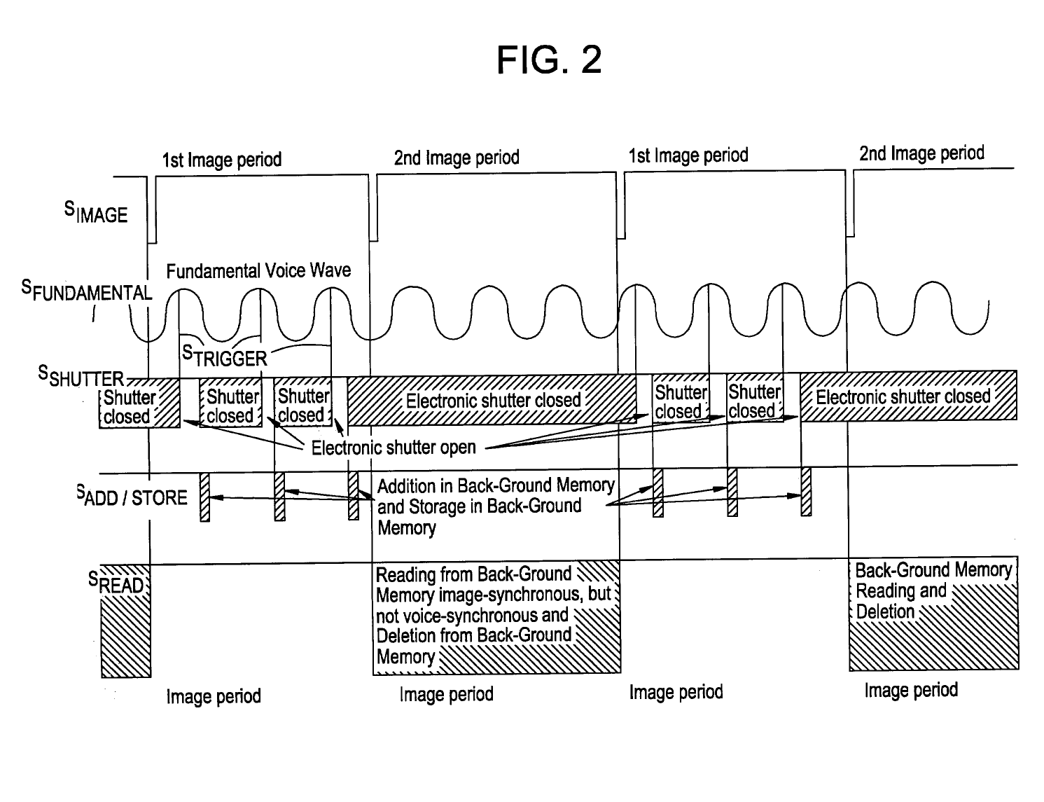

FIG. 2 a time diagram of one embodiment with signals of the device pursuant to FIG. 1 and

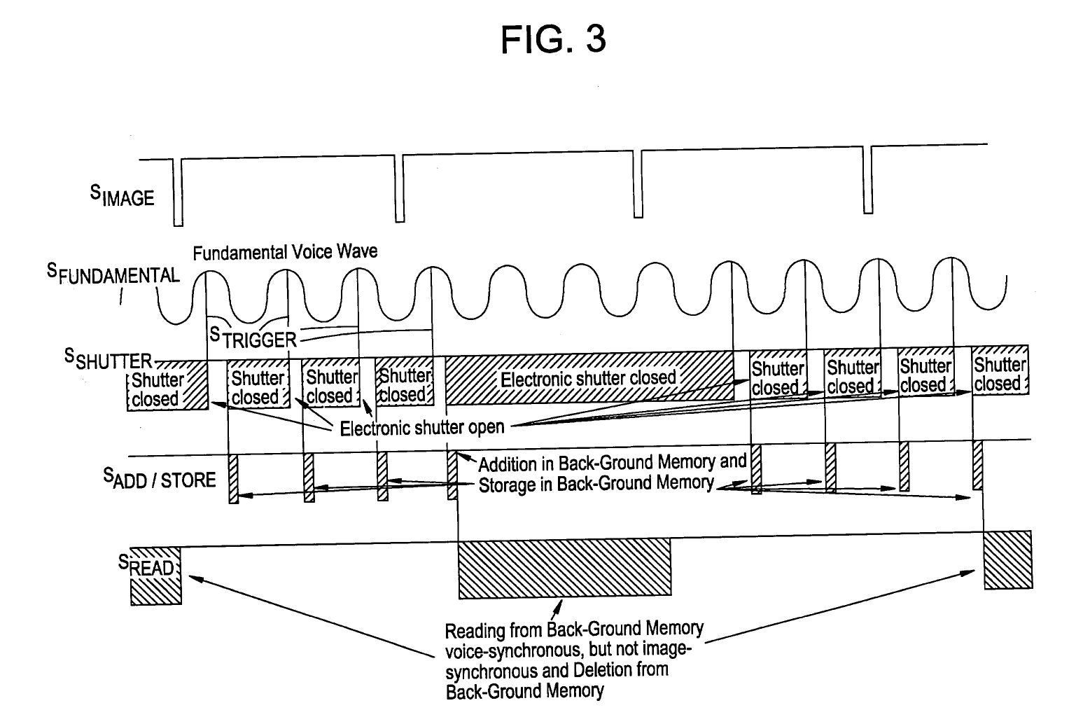

FIG. 3 a time diagram of a second embodiment with signals of the device pursuant to FIG. 1.

FIG. 1 shows the diagrammatic set-up of a device 10 for the stroboscopic recording and reproduction of images of a repetitive process 12, which is represented in the described example by the movement of vocal chords. The device 10 comprises a steady light source 14 for illuminating the process 12 that is to be observed, which is recorded by an image sensor 16 with image segments 17 and back-ground memory 18. The image sensor 16 is connected with a transducer unit 20, which transforms image information read from the back-ground memory 18 of the image sensor 16 into a video signal S_{Video}. The video signal is fed pursuant to the example to a storage device 22, which is connected with a monitor 24 for displaying the recorded images. Additionally, the storage unit 22 is connected with a reading device 26, with which the video signals that are stored in the storage device 22 can be read.

The device 10 furthermore comprises a control device 28 for controlling the image sensor 16, wherein said control device is connected on the input side with a trigger device 30, a frequency meter 32 as well as a brightness measuring device 34. The trigger device serves the generation of pulses S_{Trigger}, which are synchronous to the above-mentioned process 12 and are fed to the control device 28. To accomplish this and to record sound waves created by the vocal chords (not shown), the trigger device 30 comprises a microphone 36, which is connected with a signal processing device 38 in order to make the trigger signal S_{Trigger} available.

The frequency meter 32 is used to determine the momentary frequency of the fundamental wave of the approximately periodically repetitive process to be observed and feed it to the control device 28.

The control device 28 itself comprises an exposure control unit 40 for controlling the exposure times of the image sensor 16, an addition control device 42 for adding image information recorded by the image segments of the image sensor 16 to the image information already available in the back-ground memory 18, a memory control device 44 for storing image information sums in the back-ground memory 18 as well as a read/delete control device 46 for reading the back-ground memory 18 and the associated deletion of the back-ground memory 18.

The brightness measuring device 34 offers the possibility of measuring the brightness of images of the video signal emitted by the image sensor and/or the transducer device 20; said brightness is compared with a pre-adjusted desired brightness level, and in dependency upon the comparison of the measured brightness with the desired brightness a total exposure time of all images that are to be accumulated until the next reading process of the image sensor 16 is established.

With the help of the process's momentary frequency measured by the frequency meter 32 as well as the signal recorded by the brightness measuring device 34, the exposure times of the individual images that are to be accumulated are varied as a function of the established total exposure time and the momentary frequency of the process. Pursuant to the invention, the image sensor 16 is triggered such that even with lacking or non-periodic processes the previously established total exposure time is achieved within the current image by triggering additional exposures, which can also be asynchronous to the process.

The control device furthermore contains a time-keeping device 48 for measuring the existing actual total exposure time within the current image in order to determine whether the desired total exposure time established by the brightness measuring device 34 was achieved already after the preceding reading process of the image sensor. When the specified desired total exposure time has been reached, all subsequent exposures and addition and storage processes are blocked until the next reading process of the back-ground memory 18 of the image sensor 16 has been completed.

The storage device 22 is used to store the video signal S_{Video} supplied by the transducer device 20 while the back-ground memory of the image sensor is being read, independent of the start of the exposure of the image sensor, independent of the trigger device 30 as well as independent of the image brightness, and to issue the stored video signal during image periods in which the back-ground memory of the image sensor is not being read.

The control device 26 connected with the storage device 22 serves the purpose of controlling the storing and reading processes, which alternate with the image periods, of the image information contained in the storage.

The function of the device 10 will be explained in the following based on the time signals of two examples pursuant to FIG. 2 as well as FIG. 3.

FIG. 2 represents the time signals of a first embodiment of the invented device, wherein the reading process from the back-ground memory occurs in an image-synchronous but not voice-synchronous manner. According to the invention, the image sensor 16 is used to add the image information from several successive exposures in the image sensor 16 itself and to store it in the back-ground memory. The image sensor 16 with back-ground memory 18 is operated with an image period T_{V}, wherein the image period represents the time between two frame synchronizing pulses of the video standard that is used, e.g. PAL or NTSC. For recording an image, two image periods are required. At the beginning of the first image period, all image segments 17 as well as the back-ground memory 18 of the image sensor 16 have been deleted. The trigger signal S_{Trigger} generated by the trigger device 30 can trigger several exposures within the first image period. For this, the exposure control device 40 generates a signal S_{Shutter}, with which an electronic shutter can be controlled to open or close. At the end of each individual exposure time, the signal S_{Add/Store} selects the addition control 42 of the exposure sensor as well as the back-ground memory 18 such that new image information in the image segments is added to image information stored until then in the back-ground memory 18 and subsequently is stored again in the back-ground memory 18 through the storage control device 44.

The time-keeping device 48 establishes when the total exposure time specified by the brightness measuring device 34 has been reached. The time-keeping device 48 as well as the individual exposure times, which are varied by the control device 28, are adjusted such that in the case of a periodic process to be observed the total exposure time specified by the brightness measuring device 34 is always reached in the first image period. The above-mentioned devices are furthermore adjusted such that the exposures triggered additionally for a lacking or non-periodic process also always reach the total exposure time established by the brightness measuring device 34 in the first image period. As soon as the total exposure time specified by the brightness measuring device 34 has been reached, additional exposures are blocked.

In the subsequent second image period, image information is read from the back-ground memory 18 of the image sensor 16. An additional time-keeping device, which can for example also be included in the control device 28, eliminates the blockage of the exposures as soon as enough time for the reading process has passed.

One benefit of this method pursuant to the invention is that several exposures, which are synchronous to the process and are deduced from the fundamental voice wave S_{Fund}, are possible before reading an image from the image sensor 16. In an extreme case, for example with a fundamental voice wave with a frequency F=1 kHz and a period of 1 ms and for example for the video standard PAL with an image period of T_{V}=20 ms, 20 exposures fit into one integration time. All exposures are added to each other through the addition control unit 42 as well as the storage control unit 44 before the back-ground memory 18 of the image sensor 16 is read with the reading/deletion control unit 46. Overall, at the same length of one individual exposure, i.e. also the same pulse-duty factor and same image definition, twenty times the brightness is achieved. Even at relatively low frequencies such as F=100 Hz, brightness values twice those of the state of the art for the video standard PAL are obtained because two exposures are possible.

Since in this example the image sensor is always read only in every second frame, every other image is blank as well at the output of the image processing device 20. This problem is resolved by the connected storage device 22, which does not store blank images. The storage device 22 is additionally selected by the control device 26 such that the blank images are replaced by previously stored images.

FIG. 3 represents the time signals of one embodiment of the idea of the invention pursuant to claim 3. According to the invention, the image sensor 16 is used to add image information from several successive exposures in the image sensor 16 itself and store it in the back-ground memory. The image sensor 16 with back-ground memory 18 is operated not synchronously to the image period of the video standard that is used, but synchronously to the observed process.

The number of image periods of the employed video standard that is required for one recording is variable. As in the previous example, in this embodiment also all image segments 17 as well as the back-ground memory 18 of the image sensor 16 are deleted at the beginning of a recording. The device for example is adjusted such that the trigger signal S_{Trigger} generated by the trigger device 30 initially triggers an exposure exactly four times. For this, the exposure control unit 40 generates a signal S_{Shutter}, with which an electronic shutter can be selected to close or open. At the end of each exposure time, the signal S_{Add/Store} selects the addition control unit 42 of the exposure sensor as well as the back-ground memory 18 such that new image information in the image segments is added to image information stored until then in the back-ground memory 18 and subsequently stored again in the back-ground memory 18 through the storage control unit 44. When the desired number of exposures, as in this example four, has been reached, additional exposures are blocked. Subsequently the reading device 46 reads the back-ground memory 18 of the image sensor 16 and leaves the back-ground memory deleted, as in the starting state.

In this example the problem arises that the image information read from the image sensor does not correspond to the video standard that is used since the image information was not read synchronously with the image period. Instead of the storage device 22 as well as the storage control unit 26, in this case storage and a storage control device are used, which are in a position to store image information that does not conform to the standard on an intermediate basis and to issue it in accordance with the standard, synchronous to the image period of the video standard that is employed.

One advantage of employing the invention is that—as in the previous example—several exposures that are synchronous to the process can be performed. Additionally, voice-synchronous reading of the image sensor 16, connected with several exposures, even over a period of time of one or more image periods, allows the compromise between image brightness, image refresh rate as well as image definition to be optimized.

In the above description the case of a visual presentation of the vocal chords in the stationary position was taken into consideration. Pursuant to the state of the art it is also possible to film the retarded motion of the vocal chords by slightly shifting the start of the exposure time of the image sensor in relation to the peak or an otherwise specified trigger level of the sinusoidal curve.

Similarly, the image recording device, which is the object of this invention, enables the visual depiction of each repetitive phenomenon, even if it is aperiodic, wherein the signal processing module in this case is in a position to mark the occurrence of said phenomenon in order to create a pulse at that moment, which causes the exposure of the image sensor to be controlled.

1. Method for the stroboscopic recording and reproduction of images of a repetitive process (12), particularly moving vocal chords (larynx diagnostics), comprising the following procedural steps:
illuminate the process (12) to be observed with a steady light source (14),
generate trigger pulses S_{Trigger} for triggering exposures for the recording of image information of the process (12) with an image sensor (16),
adding the image information from several successive exposures in a back-ground memory of the image sensor (16) to form image information sums,
store the image information sums obtained this way in the back-ground memory (18) of the image sensor (16) read and delete the back-ground memory (18),
process the image information sums to a video signal.

2. Method pursuant to claim 1, characterized by the fact
that the recorded image information of n exposures with n>1 during a first image period is added in the back-ground memory and stored, wherein the image information sum stored in the back-ground memory is read as a reaction to the start of a subsequent second image period.

3. Method pursuant to claim 1, characterized by the fact that the recorded image information of n exposures with n>1 during several image periods is added in the back-ground memory and stored, wherein the image information sum stored in the back-ground memory is read as a reaction to the end of the n-th storage process.

4. Method pursuant to at least one of the previous claims, characterized by the fact that the brightness of the images of the video signal or of the image information sums read from the back-ground memory is measured, that the measured brightness is compared to a specified, desired brightness and that as a function of the comparison a total exposure time of all exposures performed between two reading processes of the image sensor (16) is established.

5. Method pursuant to at least one of the previous claims, characterized by the fact that the exposure times of the individual image information that is to be added are varied as a function of the specified total exposure time and a momentary frequency that is deduced from the process.

6. Method pursuant to at least one of the previous claims, characterized by the fact that in the case of lacking or non-periodic processes additional exposures are triggered in order to reach the established total exposure time of the current image.

7. Method pursuant to at least one of the previous claims, characterized by the fact that the actual total exposure time is measured and compared to the established total exposure time, that the start of an exposure as well as the subsequent addition and storage processes are blocked when the established total exposure time has been reached, and that the blockage of the exposures and the subsequent addition and storage processes is eliminated as soon as the next reading process of the back-ground memory has been completed.

8. Method pursuant to at least one of the previous claims, characterized by the fact that the trigger pulses S_{Trigger} are generated synchronously or asynchronously to the process or independently from the process so that the established total exposure time is reached.

9. Method pursuant to at least one of the previous claims, characterized by the fact that the trigger pulses S_{Trigger} are deduced from the fundamental wave of the periodic or nearly periodic process.

10. Device (10) for the stroboscopic recording and reproduction of images of a repetitive process (12), particularly moving vocal chords (larynx diagnostics), comprising:
a steady light source (14) to illuminate the process (12) to be observed,
a trigger device (30) for generating trigger pulses, an image sensor (16) with image segments (17)
a control device (28) with an exposure control device (40), an addition control unit (42), a storage control unit (44) as well as a reading/deletion control device (46), wherein through the exposure control device (40) the start of an exposure of the image segments (17) can be controlled as a reaction to at least one of the trigger pulses as well as the end of the exposure after a specified exposure time,
a back-ground memory (18), in which through the addition/storage control devices (42, 44) image information can be added and/or stored, wherein as a reaction to the end of an exposure time the image information that has been accumulated as a result of the exposure on the surface of the image sensor (16) in the image segments (17) is added to the image information sums stored until then in the back-ground memory (18) of the image sensor (16),
a transducer device (20), which is connected with the a image sensor (16) and which transforms the image information read from the back-ground memory (18) of the image sensor (16) into a video signal S_{Video}.

11. Device pursuant to claim 10, characterized by the fact that the device (10) contains a brightness measuring device (34) for measuring the brightness of the images of the video signal S_{Video} or of the image information sums read from the back-ground memory, wherein as a function of the measured brightness of the image the total exposure time of all image information that is to be added is established.

12. Device pursuant to claim 10 or 11, characterized by the fact that the device (10) contains a frequency meter (32) for determining the momentary frequency of the fundamental wave of the momentarily approximately periodic repetitive process (12) to be observed.

13. Device pursuant to at least one of the previous claims, characterized by the fact that the control unit (28) is connected on the input side with the brightness measuring device (34) as well as with the frequency meter (32), wherein as a function of the established total exposure time and the measured momentary frequency of the process the individual exposure times of the image information that is to be added can be varied.

14. Device pursuant to at least one of the previous claims, characterized by the fact that the control unit (28) contains a time-keeping device (48) for determining the actual total exposure time for a current image.

15. Device pursuant to at least one of the previous claims, characterized by the fact that the device contains a storage device (22) for storing the video signal S_{Video}, wherein the storage device (22) stores at least the image information read within an image period T_{V} of the image sensor (16).

16. Device pursuant to at least one of the previous claims, characterized by the fact that the device (10) contains units (26), which are in a position alternately with each image to read the storage-devices (22) and send the read video signal to the output and/or to send the video signal S_{Video} emitted by the transducer unit (20) directly to the output.

17. Device pursuant to at least one of the previous claims, characterized by the fact that the trigger device (30) contains a microphone (36) for converting acoustic vibrations of the process (12) into electric oscillations and that the trigger device (30) contains a signal processing device (38), which is connected to the microphone (36) and enables the electric oscillations to be analyzed in order to trigger a trigger pulse in each period of the electric oscillations.
